(19) United States
(12) Patent Application Publication
(10) Pub. No.: US 2019/0216984 A1
(43) Pub. Date: Jul. 18, 2019

(54) Vascularized full thickness tissue-engineered skin assembled by hydrogel, nanofibrous scaffolds and skin cell layers and preparation method thereof

(71) Applicant: The Fourth Military Medical University, Xi'an (CN)

(72) Inventors: Xueyong Li, Xi'an (CN); Hongjun Wang, Xi'an (CN); Rong Huang, Xi'an (CN); Jinqing Li, Xi'an (CN); Lirong Xu, Xi'an (CN); Yongqian Bian, Xi'an (CN); Congying Zhao, Xi'an (CN); Xiaoli Xu, Xi'an (CN); Yuejun Li, Xi'an (CN); Jing Li, Xi'an (CN)

(73) Assignee: The Fourth Military Medical University

(21) Appl. No.: 16/359,528

(22) Filed: Mar. 20, 2019

(30) Foreign Application Priority Data
Jul. 26, 2018 (CN) 201810837874.0
Nov. 8, 2018 (CN) 201811326186.4

(51) Int. Cl. A61L 27/60; C12N 5/00; C12N 5/071; C12N 5/077; A61L 27/52; C12M 1/12; D01D 5/00

(52) U.S. Cl. CPC A61L 27/60; C12N 5/0062; C12N 5/0698; C12N 5/0656; C12N 2533/90; A61L 27/52; C12M 25/14; D01D 5/0015; C12N 2533/56; C12N 5/0629

(57) ABSTRACT

A vascularized full thickness tissue engineered skin assembled by hydrogel, nanofibrous scaffolds and skin cell layers and a preparation method thereof relate to a technical field of polymer materials and biomedical materials. The artificial tissue engineered skin includes an epidermis layer and a dermis layer. The epidermal layer is formed by alternately stacking upper nanofibrous scaffolds located above the dermis layer and a kind of seed cells. The dermis layer is formed by lower nanofibrous scaffolds, the hydrogel layer above the lower nanofibrous scaffolds, and three kinds of seed cells distributed on surfaces of the lower nanofibrous scaffolds as well as inside and on a surface of the hydrogel layer. The artificial tissue engineered skin is prepared by a combination of electrospinning technology, polymer complexation technology and fiber/cell layer-gel layer-fiber/cell layer self-assembly technology. The bio-functional artificial tissue engineered skin can be used for regeneration and repair of various tissues.

### CROSS REFERENCE OF RELATED APPLICATION

The present invention claims priority under 35 U.S.C. 119(a-d) to CN 201811326186.4, filed Nov. 8, 2018.

### BACKGROUND OF THE PRESENT INVENTION

### Field of Invention

The present invention relates to a technical field of polymer materials and biomedical materials, and more particularly to a vascularized full thickness tissue engineered skin assembled by hydrogel, nanofibrous scaffolds and skin cell layers and a preparation method thereof.

### Description of Related Arts

As a complex and largest organ of the human body, the skin has complex functions such as maintaining the physiological balance of internal organs, protecting the body from infection and destruction, regulating water and body temperature, and touch. Chemical and thermal burns, contusions and cuts can cause skin damage and destroy its integrity as a barrier. For shallow and small-area injuries, the skin can be self-repaired quickly, but for deep and large-area open wounds, it is very difficult to fill only by the granulation tissue produced by fibroblasts, and its re-epithelialization is also a difficult problem to be solved in clinical practice.

Conventionally, it has been proved that artificial skin prepared by tissue engineered technology can partially or completely replace autologous skin transplantation, and it is one of the most promising methods for repairing large-area and deep-defect wounds. Commercially available artificial skin is controlled by a few developed countries such as the United States and the European Union. The product types are: skin substitutes (Integra®, AlloDerm®, Biobrane®) and tissue engineered skin (Dermagraft-TM®, Dermagraft-TC®, Epicel®, Apligraft®, etc.).

Tissue engineered skin without blood vessel or capillary is easy to fall off after transplantation, and it is not easy to quickly combine with the wound of the patient, resulting in unsuccessful transplantation. Therefore, most tissue engineered skin can only act as temporary covering or temporary substitute for skin, which cannot effectively promote wound healing to address the current need of treating large areas of skin damage. Functional capillaries are the basis for constructing tissue engineered organs for transplantation. However, stable functional tissue engineered capillaries with basement membrane have not been successfully constructed at home and abroad, so the construction of functional tissue engineered capillaries has become a major obstacle to tissue engineering.

Conventionally, in the field of artificial tissue engineered skin scaffolds, materials for skin scaffold preparation are roughly divided into two categories: one is natural biological derived materials such as collagen, chitosan, hyaluronic acid, carboxymethyl chitosan and silk fibroin; the other type is synthetic biopolymer materials, mainly polyester materials such as polyglycolide, polycaprolactone, polyhydroxyalkanoate and polycarbonate. Most skin scaffolds are prepared with biomaterials through electrospinning. Although the nanofibers obtained by electrospinning provide a suitable surface morphology for cell adhesion and growth, and facilitates the adhesion and growth of cells on the scaffold, the skin scaffold prepared by electrospinning is not conducive to the migration and proliferation of cells in the depth direction due to the small pore size, and it is difficult to achieve effective regulation of cell distribution on the fiber scaffold, which limits the application of the artificial skin scaffold in the field of skin lesion medicine.

Although many researchers have developed a variety of artificial tissue engineered skin, most of the matrix is collagen gel or sponge. Conventionally, there is no known preparation of nanofibrous scaffolds and hydrogels with pure natural polymers through layer-by-layer self-assembly techniques. Chinese patent applications “201510631809.9” and “201610793440.6” respectively report a preparation method of high-strength and high-toughness hydrogel nanofibers and a tissue engineered skin constructed by a sodium alginate hydrogel stent, but there is no related research on wound healing effects. Chinese patent applications “201610499353.X” and “201611008057.1” respectively report a micro-nano composite double-layer skin scaffold and a preparation method thereof, and a flexible artificial skin and a preparation method thereof, but there is no significantly improvement cell distribution in tissues or related research on wound healing promoting effects.

### SUMMARY OF THE PRESENT INVENTION

An object of the present invention is to provide a vascularized full thickness tissue engineered skin assembled by hydrogel, nanofibrous scaffolds and skin cell layers and a preparation method thereof for overcoming the above defects.

Accordingly, in order to accomplish the above object, the present invention provides:

a vascularized full thickness tissue engineered skin assembled by hydrogel, nanofibrous scaffolds and skin cell layers, comprising: multiple skin cell layers; multiple layers of porous nanofibrous scaffolds with a three-dimensional structure; and a single hydrogel layer;

wherein a structure of the tissue engineered skin consists of an epidermis layer and a dermis layer from top to bottom:

wherein the epidermal layer is formed by alternately stacking upper nanofibrous scaffolds located above the dermis layer and a kind of seed cells, and the seed cells are distributed between surfaces of the upper nanofibrous scaffolds and adjacent upper nanofibrous scaffolds;

wherein the dermis layer is formed by lower nanofibrous scaffolds, the hydrogel layer above the lower nanofibrous scaffolds, and three kinds of seed cells distributed on surfaces of the lower nanofibrous scaffolds as well as inside and on a surface of the hydrogel layer; wherein two of the three kinds of the seed cells are inoculated inside and on the surface of the hydrogel layer, and the other kind of the seed cells is inoculated on the surfaces of the lower nanofibrous scaffolds.

Preferably, the seed cells in the epidermis layer are keratinocytes; the three kinds of the seed cells in the dermis layer are circulating fibroblasts, vascular endothelial cells and fibroblasts, wherein the circulating fibroblasts and the vascular endothelial cells are inoculated inside and on the surface of the hydrogel layer, and the fibroblasts are inoculated on the surfaces of the lower nanofibrous scaffolds.

Preferably, the nanofibrous scaffolds are prepared with polycaprolactone, β-cyclodextrin and protein.

More preferably, type I collagen/gelatin is encapsulated in the β-cyclodextrin.

Preferably, an oxygen permeability of the nanofibrous scaffolds is 50%-60%, and a nanofiber diameter of the nanofibrous scaffolds is 200-600 nm.

Preferably, the hydrogel layer has a thickness of 0.5-1 mm and an equilibrium swelling ratio of 200%-300%.

The present invention also provides a preparation method of a vascularized full thickness tissue engineered skin assembled by hydrogel, nanofibrous scaffolds and skin cell layers, comprising steps of:

1) preparing vascular endothelial cells, circulating fibroblasts and keratinocytes;

2) co-culturing the keratinocytes with multiple layers of nanofibrous scaffolds;

wherein the step 2) specifically comprises steps of:

2-1) preparing the nanofibrous scaffolds by electrospinning with a culture dish as a receiver;

2-2) inoculating the keratinocytes in the culture dish;

2-3) repeating the steps 2-1) and 2-2) until a preset number of inoculation layers is completed; and

2-4) co-culturing the nanofibrous scaffolds with the cells in the culture dish to obtain an epidermal membrane;

3) co-culturing the vascular endothelial cells, the circulating fibroblasts and a hydrogel layer;

4) co-culturing fibroblasts with a layer of the nanofibrous scaffolds;

wherein the step 4) specifically comprises steps of:

4-1) preparing the nanofibrous scaffolds by electrospinning with the culture dish as the receiver; and

4-2) inoculating the fibroblasts in the culture dish for co-culturing;

5) paving a product cultured in the step 3) on a product cultured in the step 4); and

6) superimposing a surface of a product prepared in the step 5) with the epidermal membrane prepared in the step 2), and co-culturing until the vascularized full thickness tissue engineered skin assembled by the hydrogel, the nanofibrous scaffolds and the skin cell layers is formed.

Preferably, in the steps 2-1) and 4-1), preparing the nanofibrous scaffolds by electrospinning is specifically executed under conditions of: in an ultra-clean workbench, a DC voltage applied during an electrospinning process is 10-35 kV; a distance between a needle and the culture dish during the electrospinning process is 5-20 cm; during the electrospinning process, a syringe is driven by a syringe pump at a speed of 0.5-2.0 mL/h; an ambient temperature during electrospinning is 5-35° C.; a relative humidity in the electrospinning process is 20%-80%; a duration time of electrospinning is 1-5 min.

Preferably, in the steps 2-2) and 4-2), 1×10⁴-2×10⁴ cells/cm2 are inoculated with respect to an area of the nanofibrous scaffolds.

Preferably, after the step 6), the vascularized full thickness tissue engineered skin assembled by the hydrogel, the nanofibrous scaffolds and the skin cell layers is further subjected to sealing packaging and cryopreservation after sterilization.

Compared with the prior art, the present invention has the following beneficial effects:

The present invention provides the vascularized full thickness tissue engineered skin assembled by the hydrogel, the nanofibrous scaffolds and the skin cell layers, comprising the multiple layers of the nanofibrous scaffolds and the single hydrogel layer, wherein the nanofibrous scaffolds are porous and has the three-dimensional structure, so as to provide suitable surface morphology for cell adhesion and growth, which is conducive to cell adhesion and growth, and can simulate a natural skin basement membrane structure to provide a barrier to the epidermis layer and the dermis layer. A hydrogel layer cross-linking network contains a large amount of water for supplying cell nutrients. Combined with the underlying nanofibrous scaffolds, it can regulate cell growth and differentiation, so the composite scaffolds can better simulate the three-dimensional culture space required for cell growth, and is more conducive to cultivation and function maintenance of the keratinocytes and the epidermal basal cells. At the same time, the keratinocytes are divided into multiple layers, alternately stacked with the nanofibrous scaffolds, which allows the cells to be more evenly distributed throughout the artificial tissue engineered skin. The circulating fibroblasts (CFs) stabilize capillaries, regulate the synthesis of basement membrane components of VECs, and form gap junctions with VECs, so as to possess all the functions of capillary supporting cells, thus constructing biologically active vascularized tissue engineered skin substitutes. Animal experiments have shown that the artificial tissue engineered skin can significantly improve the distribution of cells in the tissue and promote wound healing. Thus, the bioactive artificial vascularized tissue engineered skin can be used for regeneration and repair of various tissues, particularly for wound healing, reduction of scar formation, skin regeneration, and the like.

### BRIEF DESCRIPTION OF THE DRAWINGS

FIG. 1 is a structural view of an artificial vascularized full thickness tissue engineered skin of the present invention;

FIG. 2-1 is a photo of a dermis layer of the tissue engineered skin of the present invention;

FIG. 2-2 is a photo of the full thickness tissue engineered skin of the present invention;

FIG. 3-1 is a scanning electron micrograph of vascular endothelial cells (VECs) and circulating fibroblasts (CFs) which are co-cultured on PCL-collagen nanofibrous scaffolds;

FIG. 3-2 is a laser scanning confocal micrograph of the vascular endothelial cells (VECs) and the circulating fibroblasts (CFs) which are co-cultured on the PCL-collagen nanofibrous scaffolds;

FIG. 4 illustrates healing test results of the artificial vascularized tissue engineered skin of the present invention and other materials applied to wound miniature pigs;

FIG. 5-1 is a Masson staining diagram of the artificial tissue engineered skin of the present invention after covering wound for 21 d;

FIG. 5-2 is a Masson staining diagram of wound margin tissues in contact with skin;

FIG. 5-3 illustrates immunofluorescence staining results of HLA-ABC (human histocompatibility antigen)-vimentin at the wound margin tissues in contact with the skin.

### DETAILED DESCRIPTION OF THE PREFERRED EMBODIMENT

Referring to the drawings of embodiments, the present invention will be further illustrated.

The present invention combines electrospinning technology, polymer complexing technology and fiber/cell layer-gel layer-fiber/cell layer self-assembly technology to prepare a vascularized tissue engineered skin with good plasticity, suitable mechanical properties, three-dimensional stereostructure with high porosity, and bio-functions. The present invention adopts electrospinning technology to process polycaprolactone-β-CD-collagen/gelatin nanofibrous scaffolds at a nanometer level into an interpenetrating porous three-dimensional microstructure which is suitable for growth and differentiation of keratinocytes, and can be coated with various cytokines or drugs as needed. The layer-by-layer self-assembly (LBL) technology is also used to carry out fiber-cell layer stacking, which promotes cell attachment and growth in various aspects and improves the efficiency of cell therapy. At the same time, in combination with circulating fibroblasts and vascular endothelial cells, blood vessels are formed in the hydrogel, and then layers of fibroblasts and fibrous scaffolds are superposed to form a vascularized dermis layer.

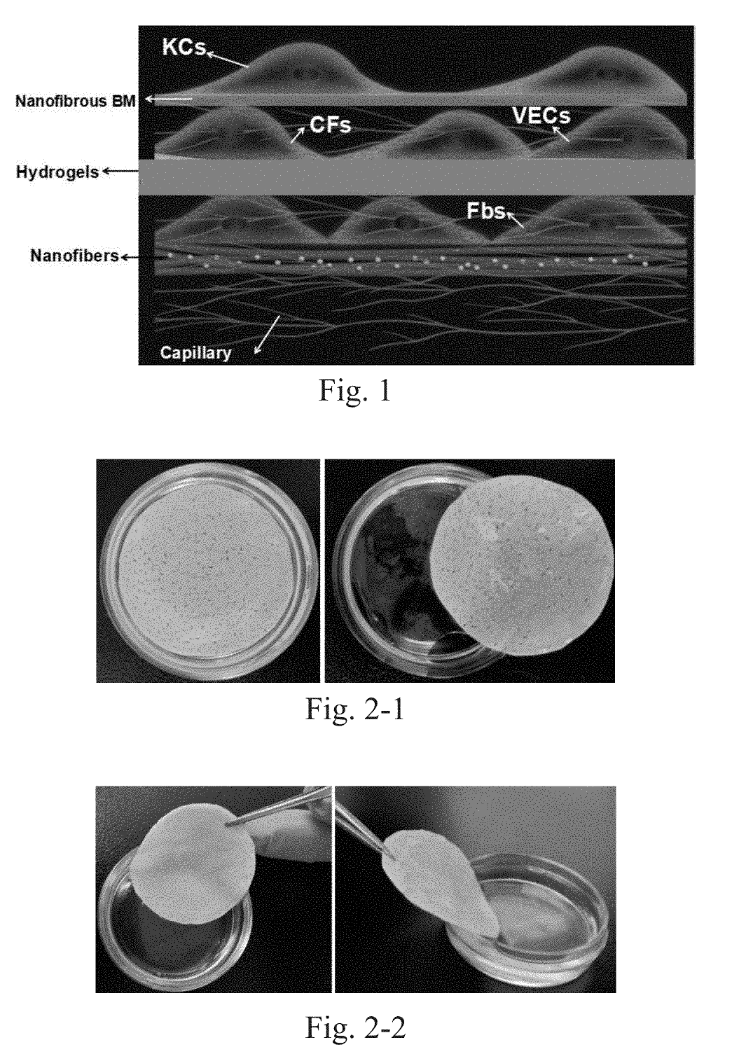

As shown in FIG. 1, keratinocytes were inoculated on polycaprolactone-β-CD/collagen nanofibrous scaffolds, and nanofibrous scaffolds and cells were superposed layer by layer to form an epidermal membrane with layer-by-layer self-assembly technique. Vascular endothelial cells and circulating fibroblasts are inoculated in the hydrogel to form blood vessels, and fibroblasts are inoculated in the underlying nanofiber scaffold to form the artificial vascularized tissue engineered skin. Separation and culture method of keratinocytes, circulating fibroblasts and vascular endothelial cells are as follows.

### Embodiment 1: Separation and Purification of Keratinocytes

The foreskin was clarified by repeated washing with sterile PBS several times until the PBS is clear. The foreskin is cut into strips after removing other tissues such as fascia; then Dispase is added before digestion overnight in a refrigerator at 4° C. The foreskin that had been digested overnight was rewarmed at 37° C. Then epidermis and dermis were separated, and the isolated epidermis was washed in PBS. The isolated epidermis was added to trypsin (containing EDTA) and digested at 37° C. The digested juice was filtered through a sieve, and the filtrate was collected and centrifuged. After washing with PBS, the cells were resuspended in a K-SFM medium, and the cells were inoculated at a cell density of 1×10⁵/cm² and cultured in a 37° C. incubator. After 2 days, the liquid was changed for the first time, and then the liquid was changed every 3 days.

### Embodiment 2: Separation and Purification of Circulating Fibroblasts

A lymphocyte separation solution was added to an L tube, and the mixture was centrifuged to the lower layer. Blood was diluted with PBS, and then was added to the L tube for centrifugation separation. The upper layer of plasma was discarded, and the leucorrhea layer was aspirated and added to a centrifuge tube to be diluted with PBS and centrifuged. The above procedures were repeated 3 times. Subsequently, the cells were counted after resuspending in PBS, and inoculated in a culture dish at a density of 1×10⁶/mL.

### Embodiment 3: Separation and Purification of Vascular Endothelial Cells

The HUVEC cell line purchased from Sciencell was resuscitated at 37° C., and inoculated into a 100 mm culture dish. After the culture dish was full of cells, trypsin was used for passage digestion.

### Embodiment 4: Preparation of Polycaprolactone-β-CD-Collagen/Gelatin Nanofibrous Scaffolds

In an ultra-clean workbench, polycaprolactone (PCL) was dissolved in a N,N-dimethylformamide-dichloromethane mixed solution with a mass ratio of 1:(1-3), so as to obtain a 8 wt % polycaprolactone solution; β-CD was added to the polycaprolactone solution, so that the mass fraction of β-CD was 0.1-1% before heating to 50-70° C. and stirring evenly with a stirring time of 6-12 hours. As a result, a polycaprolactone-β-CD solution was obtained, wherein β-CD may be pre-encapsulated with collagen/gelatin.

The obtained polycaprolactone-β-CD-collagen/gelatin solution was sucked into a metal needle-equipped plastic syringe of an electrospinning apparatus in the ultra-clean workbench. The syringe needle was 10 cm from the culture dish as a collector, and a DC voltage applied was 20 kV. The syringe was driven by a syringe pump at a rate of 1.0 mL/h, with an ambient temperature of 25° C., a relative humidity of 40%, and an electrospinning time of 3-5 min, so as to obtain the nanofibrous scaffolds, which were received in the culture dish.

### Embodiment 5: Preparation of Polycaprolactone-β-CD Collagen/Gelatin Nanofibrous Scaffolds

Referring to the method of the embodiment 4, in the polycaprolactone-β-CD-collagen/gelatin solution, the mass concentration of polycaprolactone has 4%; the mass concentration of β-CD was 0.02-0.1%; the distance between the needle and the culture dish was 20 cm, the DC voltage applied was 10 kV; the syringe was driven by the syringe pump at a rate of 2.0 mL/h, the ambient temperature was 35° C., the relative humidity was 20%, and the electrospinning time was 5 min.

### Embodiment 6: Preparation of Polycaprolactone-β-CD Collagen/Gelatin Nanofibrous Scaffolds

Referring to the method of the embodiment 4, in the polycaprolactone-β-CD-collagen/gelatin solution, the mass concentration of polycaprolactone has 12%; the mass concentration of β-CD was 0.2-1%; the distance between the needle and the culture dish was 5 cm, the DC voltage applied was 35 kV; the syringe was driven by the syringe pump at a rate of 0.5 mL/h, the ambient temperature was 5° C., the relative humidity was 80%, and the electrospinning time was 1 min.

### Embodiment 7

(1) inoculating fibroblasts on surfaces of polycaprolactone-β-CD-collagen/gelatin nanofibrous scaffolds with layer-by-layer self-assembly technique (LBL technology) for co-culturing;

wherein in the ultra-clean workbench, the nanofibrous scaffolds received in a 60 mm culture dish were prepared according to the methods of embodiments 4-6; the fibroblasts were inoculated in the culture dish with the nanofibrous scaffolds, and the inoculated cell density was 1×10⁴-1×10⁵ cells/mL; the culture dish was placed in a 37° C. CO₂ incubator, and a single layer of nanofiber scaffold-fibroblast complex can be formed by culturing.

(2) alternately inoculating keratinocytes on the surface of the polycaprolactone-β-CD-collagen/gelatin nanofibrous scaffolds with the layer-by-layer self-assembly technique (LBL technology) for layer-by-layer three-dimensional co-culturing;

wherein in the ultra-clean workbench, the nanofibrous scaffolds received in a 60 mm culture dish were prepared according to the methods of embodiments 4-6; the keratinocytes obtained in the embodiment 1 were inoculated in the culture dish with the nanofibrous scaffolds, and the inoculated cell density was 1×10⁴-1×10⁵ cells/mL; then the culture dish was used as a receiver of the nanofibrous scaffolds for electrospinning before cell inoculation; thus, cells were inoculated to 5 layers per layer. Since the whole process took place on the surface of the medium, the cells remained hydrated in the assembly process, so as to form a total of 5 layers of cells/nanofibers alternately layered three-dimensional structures. The culture dish was placed in a 37° C. CO₂ incubator, and a K-SFM supplement medium was added; the culture was continued for 1-2 weeks to form a layer-by-layer self-assembled artificial epidermal membrane.

### Embodiment 8

Referring to the preparation method of the embodiment 7 (2), wherein the culture dish had a diameter of 100 mm, the number of cells inoculated per layer was 1×10⁶ cells, and a total of 20 layers were inoculated.

### Embodiment 9: Preparation of Hydrogel-Cell Complex

The three-dimensional collagen was mixed with VECs and CFs. The three-dimensional collagen was prepared as: NaHCO₃ and L-Glutamic were added to a M199 medium, and after mixing the Collagen Type I, the pH was adjusted with NaOH; the product was placed on ice for subsequent use.

A Basal Medium mixed medium was prepared, and the VECs and CFs were digested and resuspended in the Basal Medium mixed medium. The cell suspension was mixed with the three-dimensional collagen, spread on a fiber scaffold, and incubated at 37° C.

### Embodiment 10: Preparation of Full Thickness Tissue Engineered Skin

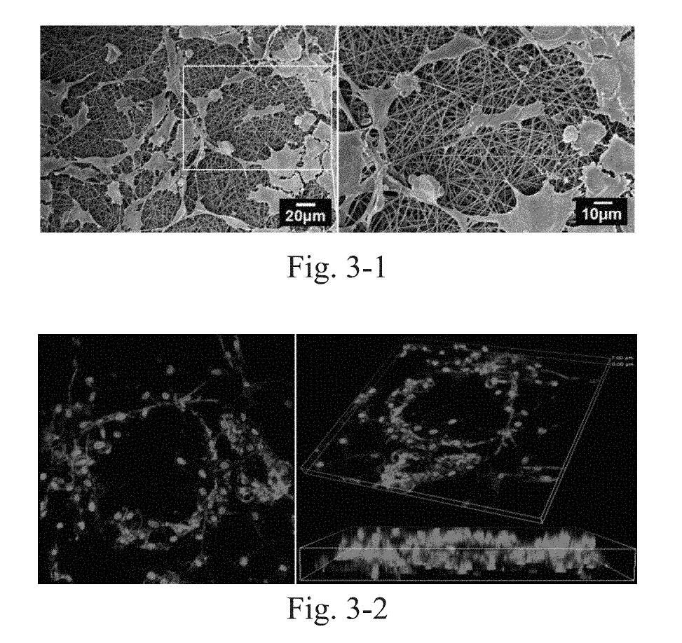

The fibroblast-inoculated fiber scaffold prepared in the embodiment 7 (1) was placed under the cell-three-dimensional collagen mixture prepared in the embodiment 9, and then the epidermal membrane prepared in the embodiment 7 (2) was superposed on the cell-three-dimensional collagen mixture; the three-layer complex was incubated at 37° C. and refilled for subsequent use. Image acquisition was performed on the dermis and full thickness skin (see FIGS. 2-1 and 2-2).

### Embodiment 11

The artificial vascularized tissue engineered skin prepared in the embodiment 10 with layer-by-layer self-assembly was sealed and packaged after ethylene oxide sterilization, and had a cryoprotectant in the packaging bag; then the product was frozen after packaging; the engineered skin after packaging can be frozen in a refrigerator at −78° C. to −82° C. or in liquid nitrogen.

### Embodiment 12

Vascular endothelial cells and circulating fibroblasts were inoculated on the polycaprolactone-β-cyclodextrin nanofibrous scaffolds for 3 days, then fixed in glutaraldehyde solution at 4° C. overnight before washing with PBS, dehydration by ethanol with a series of gradients, and then vacuum freeze-drying; after drying for 6 hours, vacuum carbonation was performed, and the growth of the cells on the fiber surface was observed by field emission scanning electron microscopy.

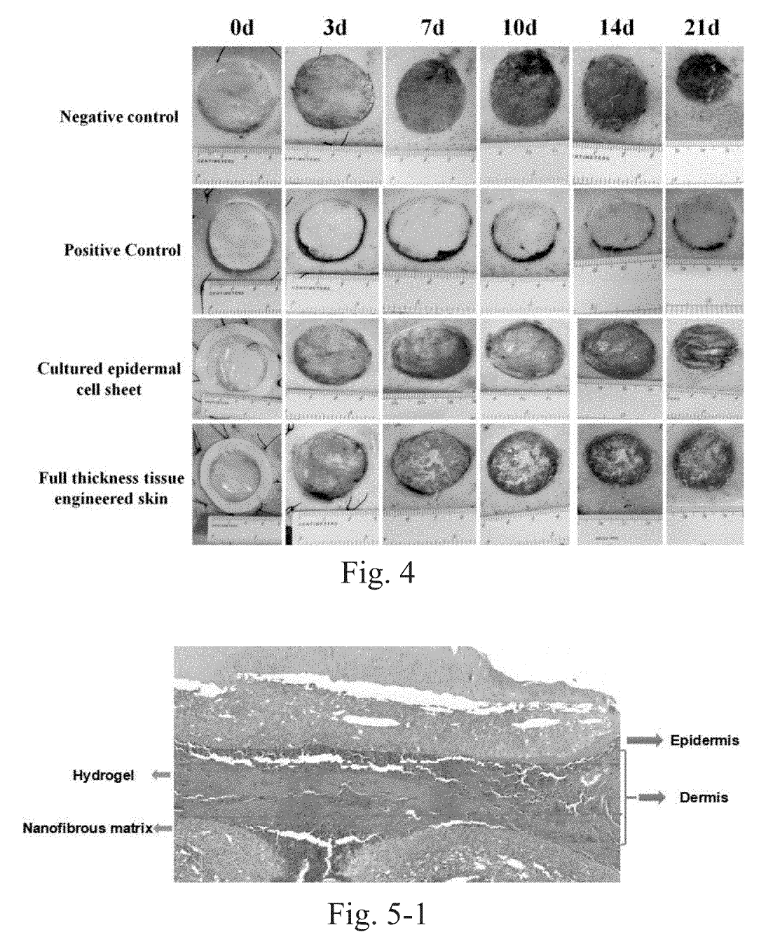

FIG. 3-1 is a scanning electron micrograph of vascular endothelial cells (VECs) and circulating fibroblasts (CFs) which are co-cultured on PCL-collagen nanofibrous scaffolds; and FIG. 3-2 is a confocal micrograph thereof. The results showed that the co-cultured cells formed a lumen, and the diameter of the lumen satisfied a requirement of 5-20 μm.

### Embodiment 13

2-month-old Bama miniature pigs with a weight of 15-20 kg were chosen, which are half male and half female of SPF grade, provided by the Animal Center of the Fourth Military Medical University of China. They were randomly divided into model groups and control groups according to a random number table method, wherein there were 4 in each group and a total of four groups (a negative control group, a positive control group, an artificial epidermal cell sheet group and an artificial tissue engineered skin group), and each group was fasted 12 h before surgery. Then intramuscular anesthesia was performed with 2% pentobarbital sodium with skin preparation on the back, iodophor disinfection of the back skin, and a sterile towel. On two sides of back midline of each pig, two round full thickness skin defect wounds reaching a muscle fascia with a diameter of 3 cm were cut out, and 8 wounds were cut on the back of each pig.

Negative control group: a single layer of oil yarn covered the wounds; Positive control group: autologous whole skin was grafted;

Artificial epidermal cell sheet group: artificial epidermal membrane was grafted, wound surface was covered with Vaseline oil yarn and pressure bandage;

Full thickness vascularized tissue engineered skin group: full thickness vascularized tissue engineered skin was grafted, wound surface was covered with Vaseline oil yarn and pressure bandage.

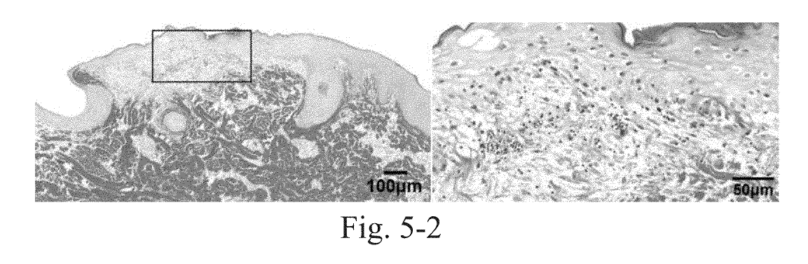

According to the grouping, the covering material is cut according to the size of the wound surface for covering the wound surface. Sampling time was based on the wound healing time of each group. After the wound was formed, the wound healing was monitored in real time in different cycles. Image analysis software was used to analyze photos before and after wound treatment. A healing rate of greater than 90% was judged as healing. The experimental results are shown in FIG. 4; wherein there are significant differences in the effect of different treatments on wound healing. Compared with the negative control group (conventional yarn covering group) and other groups, epithelialization degree of the artificial vascularized tissue engineered skin prepared by the present invention was higher after the wound was healed, indicating that the such material promoted the wound healing best.

### Embodiment 14

On the basis of the embodiment 13, after the wound was completely healed, new tissues (with wound margin) were taken, embedded in paraffin, sliced, and detected by Masson staining. After the wound is completely healed, the new tissues (with the wound margin) were taken, embedded in paraffin, sliced for baking, dewaxed, gradient-added into water and rinsed, and then stained with R1, R2, R3, R4 (Biyuntian staining kits) in sequence and sealed with neutral gel; collagen fibers, blood vessels and other skin structures in the tissues were observe with a fluorescence microscope.

The results of the test are shown in FIGS. 5-1 and 5-2. The results of Masson staining show that the artificial tissue engineered skin prepared by the present invention has excellent in vivo degradation properties and has neovascularization after material implantation. The experimental results confirmed that the artificial tissue engineered skin prepared by the present invention has better in vivo degradation performance in the same implantation cycle, and neovascularization can be observed inside the fiber.

### Embodiment 15

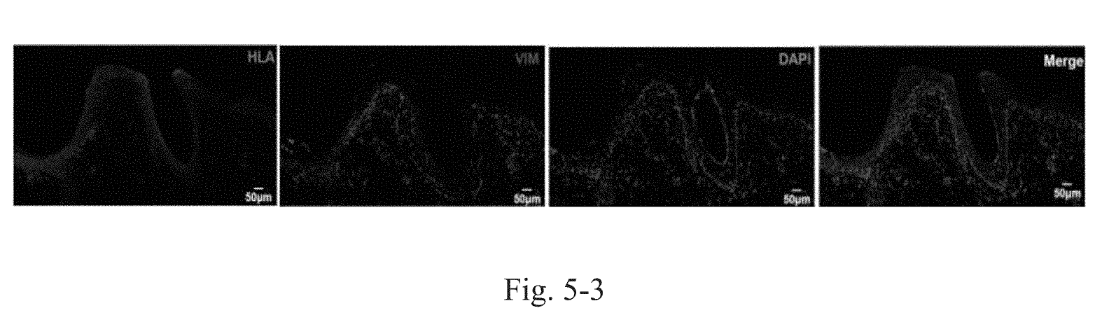

Tissues in contact with the tissue engineered skin were washed 3 times with PBS, fixed in 4% formaldehyde solution (dissolved in PBS) for 30 min, dried for 5 min, then washed 3 times with PBS, permeated with 0.5% TRITON X-100 for 20 min, and washed with PBS. Cells were stained with HLA, Vimentin and DAPI at room temperature, wherein unbound staining solution was removed with PBS, and the cells are mounted for confocal observation. The results of the experiment are shown in FIG. 5-3, wherein red is the labeled human source cell and green is the labeled endogenous fibroblast, wherein nuclei are marked blue by DAPI staining. The results show that the artificial full thickness tissue engineered skin of the present invention has good biocompatibility in the body, rarely causes an inflammatory reaction, and interacts with endogenous cells.

In summary, the present invention utilizes the layer-by-layer technique of cell and nanofiber for the first time to construct functional capillaries by CFs and VECs, and to prepare a tissue engineered full thickness skin with functional capillary and good mechanical properties, so as to finally form a vascularized tissue engineered skin for clinical use. The vascularized tissue engineered skin can be applied to II and III degrees of wounds, deep burn residual wounds, donor areas, skin graft areas, friction wounds, mechanical wounds, ulcers or diabetic refractory wounds, etc., which can quickly close wounds, so as to reduce the risk of infection and prevent the exposure of organs, as well as significantly improve the quality of skin healing. It is expected to solve the conventional treatment problems of deep and large-area open wounds, and greatly promote the progress and industrialization of tissue engineered skin.

What is claimed is:

1. A vascularized full thickness tissue engineered skin assembled by hydrogel, nanofibrous scaffolds and skin cell layers, comprising: multiple skin cell layers; multiple layers of porous nanofibrous scaffolds with a three-dimensional structure; and a single hydrogel layer;
wherein a structure of the tissue engineered skin consists of an epidermis layer and a dermis layer from top to bottom:
wherein the epidermal layer is formed by alternately stacking upper nanofibrous scaffolds located above the dermis layer and a kind of seed cells, and the seed cells are distributed between surfaces of the upper nanofibrous scaffolds and adjacent upper nanofibrous scaffolds;
wherein the dermis layer is formed by lower nanofibrous scaffolds, the hydrogel layer above the lower nanofibrous scaffolds, and three kinds of seed cells distributed on surfaces of the lower nanofibrous scaffolds as well as inside and on a surface of the hydrogel layer; wherein two of the three kinds of the seed cells are inoculated inside and on the surface of the hydrogel layer, and the other kind of the seed cells is inoculated on the surfaces of the lower nanofibrous scaffolds.

2. The vascularized full thickness tissue engineered skin, as recited in claim 1, wherein the seed cells in the epidermis layer are keratinocytes; the three kinds of the seed cells in the dermis layer are circulating fibroblasts, vascular endothelial cells and fibroblasts, wherein the circulating fibroblasts and the vascular endothelial cells are inoculated inside and on the surface of the hydrogel layer, and the fibroblasts are inoculated on the surfaces of the lower nanofibrous scaffolds.

3. The vascularized full thickness tissue engineered skin, as recited in claim 1, wherein the nanofibrous scaffolds are prepared with polycaprolactone, β-cyclodextrin and protein.

4. The vascularized full thickness tissue engineered skin, as recited in claim 3, wherein type I collagen/gelatin is encapsulated in the β-cyclodextrin.

5. The vascularized full thickness tissue engineered skin, as recited in claim 1, wherein an oxygen permeability of the nanofibrous scaffolds is 50%-60%, and a nanofiber diameter of the nanofibrous scaffolds is 200-600 nm.

6. The vascularized full thickness tissue engineered skin, as recited in claim 1, wherein the hydrogel layer has a thickness of 0.5-1 mm and an equilibrium swelling ratio of 200%-300%.

7. A preparation method of a vascularized full thickness tissue engineered skin assembled by hydrogel, nanofibrous scaffolds and skin cell layers, comprising steps of:
1) preparing vascular endothelial cells, circulating fibroblasts and keratinocytes;
2) co-culturing the keratinocytes with multiple layers of nanofibrous scaffolds;
wherein the step 2) specifically comprises steps of:
2-1) preparing the nanofibrous scaffolds by electrospinning with a culture dish as a receiver;
2-2) inoculating the keratinocytes in the culture dish;
2-3) repeating the steps 2-1) and 2-2) until a preset number of inoculation layers is completed; and
2-4) co-culturing the nanofibrous scaffolds with the cells in the culture dish to obtain an epidermal membrane;
3) co-culturing the vascular endothelial cells, the circulating fibroblasts and a hydrogel layer;
4) co-culturing fibroblasts with a layer of the nanofibrous scaffolds;
wherein the step 4) specifically comprises steps of:
4-1) preparing the nanofibrous scaffolds by electrospinning with the culture dish as the receiver; and
4-2) inoculating the fibroblasts in the culture dish for co-culturing;
5) paving a product cultured in the step 3) on a product cultured in the step 4); and
6) superimposing a surface of a product prepared in the step 5) with the epidermal membrane prepared in the step 2), and co-culturing until the vascularized full thickness tissue engineered skin assembled by the hydrogel, the nanofibrous scaffolds and the skin cell layers is formed.

8. The preparation method, as recited in claim 7, wherein in the steps 2-1) and 4-1), preparing the nanofibrous scaffolds by electrospinning is specifically executed under conditions of:
in an ultra-clean workbench, a DC voltage applied during an electrospinning process is 10-35 kV; a distance between a needle and the culture dish during the electrospinning process is 5-20 cm; during the electrospinning process, a syringe is driven by a syringe pump at a speed of 0.5-2.0 mL/h; an ambient temperature during electrospinning is 5-35° C.; a relative humidity in the electrospinning process is 20%-80%; a duration time of electrospinning is 1-5 min.

9. The preparation method, as recited in claim 7, wherein:
in the steps 2-2) and 4-2), 1×10⁴-2×10⁴ cells/cm2 are inoculated with respect to an area of the nanofibrous scaffolds.

10. The preparation method, as recited in claim 7, wherein after the step 6), the vascularized full thickness tissue engineered skin assembled by the hydrogel, the nanofibrous scaffolds and the skin cell layers is further subjected to sealing packaging and cryopreservation after sterilization.
